(19) United States
(12) Patent Application Publication
(10) Pub. No.: US 2026/0266770 A1
(43) Pub. Date: Sep. 10, 2026

(54) FINITE ELEMENT SIMULATION (FES) AND TRANSFER LEARNING (TL)-BASED INVERSION METHOD FOR FIRMNESS OF ON-TREE PEACH FRUIT

(71) Applicant: ZHEJIANG UNIVERSITY, Hangzhou (CN)

(72) Inventors: Di CUI, Hangzhou (CN); Jiaqi XIONG, Hangzhou (CN)

(73) Assignee: ZHEJIANG UNIVERSITY, Hangzhou (CN)

(21) Appl. No.: 19/661,765

(22) Filed: Apr. 29, 2026

Related U.S. Application Data
(63) Continuation of application No. PCT/CN2025/120517, filed on Sep. 10, 2025.

(30) Foreign Application Priority Data
Sep. 29, 2024 (CN) 202411370312.1

(51) Int. Cl. G01N 29/24; G01N 29/04; G01N 33/02; G06N 3/096

(52) U.S. Cl. CPC G01N 29/2418; G01N 29/04; G01N 33/025; G06N 3/096; G01N 2291/023; G01N 2291/02827

(57) ABSTRACT

Provided is a finite element simulation (FES) and transfer learning (TL)-based inversion method for firmness of an on-tree peach fruit, including the following steps: step 1, collecting vibration signals of on-tree peach fruits at different growth stages by using a laser Doppler vibrometer, preprocessing the vibration signals to serve as a model input, measuring the reference firmness values of the peach fruits by using a texture analyzer, and constructing an experimental dataset; step 2, establishing ten finite element models with high bionic fidelity for peach fruit vibration, and calculating the simulated vibration responses; step 3, modifying material properties of the finite element models in the step 2 to obtain vibration responses of peach fruits with different firmness levels, and constructing a simulated dataset; and step 4, constructing a domain-adversarial neural network (DANN), and achieving domain transfer and firmness inversion through adversarial training.

### CROSS REFERENCE TO THE RELATED APPLICATIONS

This application is a continuation application of International Application No. PCT/CN2025/120517, filed on Sep. 10, 2025, which is based upon and claims priority to Chinese Patent Application No. 202411370312.1, filed on Sep. 29, 2024, the entire contents of which are incorporated herein by reference.

### TECHNICAL FIELD

The present disclosure relates to the field of fruit quality detection, and in particular, to a finite element simulation (FES) and transfer learning (TL)-based inversion method for firmness of an on-tree peach fruit.

### BACKGROUND

Firmness is correlated with taste, maturity, and storage tolerance of peaches, and serves as a key quality indicator for the peaches. A demand for firmness measurement runs through an entire peach industry chain. Peaches harvested within a specific firmness range usually exhibit greater damage resistance, have a longer shelf life, and offer better taste once fully softened. Therefore, pre-harvest firmness monitoring is of great significance for determining optimal harvest time of the peaches.

Traditional fruit firmness detection methods are destructive and cannot meet a demand for large-scale detection. The acoustic vibration method analyzes a vibration response of a vibrating peach fruit, extracts a vibration feature to invert a mechanical parameter of the peach fruit, and then obtains firmness of the peach fruit, which can reduce mechanical damage to the peach fruit. However, in pre-harvest applications, due to interference of natural wind, the vibration response of the peach fruit is full of noise, making it difficult for even experienced workers to accurately extract the vibration feature from a signal. At present, the deep learning-based feature extraction method performs well in vibration signal processing. It can extract a feature related to a target task from a large number of signals in an unsupervised manner, and thus has been widely used in mechanical fault diagnosis, electrocardiogram analysis, agricultural product quality detection, and other fields. However, traditional deep learning methods require a large number of data samples and assume that training data and test data have a same feature distribution, which imposes high requirements on a quantity and quality of samples. Collecting more samples increases the input of human resources, material resources, and funds, and extensive experiments will cause various deviations due to fatigue of operators. Especially in agricultural field applications, it is almost impossible to obtain such a large number of high-quality and uniformly-distributed samples. Numerical simulation provides an effective solution to this problem. Vibration simulation can be performed on an object by using a numerical simulation technique, such that a large amount of simulated data is obtained. The finite element analysis method is a commonly used numerical simulation technique. However, due to a complex shape and structure of a fruit and a difficulty in determining material properties, the existing finite element model of the fruit often has an oversimplified shape, internal structure, or material property, resulting in a significant discrepancy between simulated data of the finite element model and experimental data. Constructing an accurate finite element model can improve a similarity between the simulated data and the experimental data. Nevertheless, complexity and variability of a field measurement environment, along with diversity in a fruit size and shape, inevitably lead to a difference between feature distributions of the simulated data and the experimental data. This can reduce prediction accuracy of a deep learning prediction model. Therefore, how to achieve accurate inversion of pre-harvest firmness of the peach fruit under a small sample size remains an unsolved problem.

### SUMMARY

An objective of the present disclosure is to address the aforementioned shortcomings by providing an FES and TL-based inversion method for firmness of an on-tree peach fruit. This method overcomes a problem of low accuracy in a traditional finite element model of a fruit due to excessive simplification, resolves problems of a small number of dataset samples and a significant difference in feature distribution, and provides an effective solution for accurately inverting pre-harvest firmness of a peach fruit.

In order to resolve the above technical problem, the present disclosure provides the following technical solution: An FES and TL-based inversion method for firmness of an on-tree peach fruit includes following steps:
step 1, collecting vibration signals of on-tree peach fruits at different growth stages by using a laser Doppler vibrometer, preprocessing the vibration signals to serve as a model input, measuring reference firmness values of the peach fruits by using a texture analyzer to serve as a model output, and constructing an experimental dataset;
step 2, establishing ten finite element models with high bionic fidelity for peach fruit vibration, and calculating a simulated vibration response, which includes:
step 2.1: acquiring three-dimensional (3D) point clouds of outer surfaces of the peach fruit, a peach pit, and a peach kernel by using a handheld laser 3D scanner, and constructing a 3D geometric model with an accurate shape and structure of the peach fruit through reverse modeling;
step 2.2, testing compressive elastic moduli of peach flesh, the peach pit, and the peach kernel and a tensile elastic modulus of peach peel by using the texture analyzer and a universal mechanical testing machine respectively, to serve as material properties of the finite element models; and
step 2.3, establishing the finite element models with high bionic fidelity for the peach fruit vibration based on the 3D geometric model of the peach fruit, the material properties measured in the step 2.2, the experimental excitation, and the fruit constraint condition, and simulating and calculating a simulated vibration response at a response point;
step 3, modifying the material properties of the finite element models in the step 2 to obtain simulated vibration responses of peach fruits with different firmness levels; and
step 4, constructing a domain-adversarial neural network (DANN), performing training by using simulated data with a firmness value as a source domain and experimental data without the firmness value as a target domain, and achieving domain transfer and firmness inversion through adversarial training, which includes:
step 4.1, constructing a feature extraction network based on multi-scale convolution and a channel attention mechanism, thereby achieving adaptive feature extraction;
step 4.2, constructing a predictor, performing firmness prediction on a feature through a fully connected layer, and selecting a mean square error (MSE) as a loss function of the predictor:

$${ℒ}_{p} = \frac{{\sum }_{i = 1}^{N} {( {y}_{i} - \hat{{y}_{ι}} )}^{2}}{N}$$

where [symbol] represents the loss function of the predictor; N represents a quantity of samples; yᵢ represents the firmness value; and ŷ_{ι} represents a predicted firmness value; and
step 4.3, constructing a domain discriminator, where in this step, a gradient direction is automatically reversed during backpropagation through a gradient reversal layer, so as to maximize a domain classification loss and confuse target domain data and source domain data; and a cross-entropy loss is used as a loss function of the domain discriminator:

$${L}_{d} = \frac{1}{N} {\sum }_{i = 1}^{N} {y}_{i} log \hat{{y}_{ι}} + ( 1 - {y}_{i} ) log ( 1 - \hat{{y}_{ι}} )$$

where L_{d} represents the loss function of the domain discriminator; N represents the quantity of samples; yᵢ represents the firmness value; and ŷ_{ι} represents the predicted firmness value; and
step 4.4, forming a loss function of the network jointly by the loss function of the predictor and the loss function of the domain discriminator:

$$L = {L}_{ρ} - λ {L}_{d}$$

where L represents a total loss function; and λ represents a parameter for adjusting an adversarial intensity.

As a further improvement of the present disclosure, in a signal preprocessing process, a band-pass filter is used for filtering, and a power spectral density of the vibration response is calculated as the model input.

As a further improvement of the present disclosure, in the step 1, a force-displacement curve in a puncture process is obtained through a puncture experiment, and an initial slope of the curve is used as the reference firmness value of the peach fruit.

As a further improvement of the present disclosure, in the step 2.1, a point cloud of the peach fruit is denoised and surfaced in Geomagic software, and then imported into Solidworks software, so as to perform component combination based on a physiological structure of the peach fruit, so as to construct a complete 3D model of the peach fruit.

As a further improvement of the present disclosure, in the step 2.2, the peach flesh is prepared into a 15 mm×15 mm×15 mm cubic sample, and a compression test is carried out using the texture analyzer. After the force-displacement curve is obtained, the elastic modulus of the peach flesh is calculated according to the following formula:

$$E = \frac{σ}{ε} = \frac{FL}{A Δ L}$$

where E represents the elastic modulus, σ represents stress, ε represents strain, F represents a compressive force, L represents an initial length of the sample, A represents cross-sectional area of the sample, and ΔL represents a deformation amount.

As a further improvement of the present disclosure, in the step 2.2, the complete peach pit and peach kernel are placed on the texture analyzer for the compression test. After the force-displacement curve is obtained, the elastic moduli of the peach pit and the peach kernel are calculated according to the following formula:

$$E = {\frac{0 \text{.338} {K}_{U}^{3 / 2} F ( 1 - {μ}^{2} )}{{D}^{3 / 2}} [ {{K}_{U} ( \frac{1}{{R}_{U}} + \frac{1}{{R}_{U}^{′}} )}^{1 / 3} + {{K}_{L} ( \frac{1}{{R}_{L}} + \frac{1}{{R}_{L}^{′}} )}^{1 / 3} ]}^{\frac{3}{2}}$$

where E represents the elastic modulus, F represents a force, D represents deformation, K_{U} and K_{L} represent constants determined by curvatures of contact points on upper and lower surfaces, R_{U} and R′_{U} represent maximum and minimum curvature radii of the contact point on the upper surface, and R_{L} and R′_{L} represent maximum and minimum curvature radii of the contact point on the lower surface.

As a further improvement of the present disclosure, in the step 2.2, the peach peel is prepared into a rectangular sample, and a tensile test is conducted on the peach peel by using the universal mechanical testing machine. The elastic modulus of the peach peel is calculated according to the following formula:

$$E = \frac{σ}{ε} = \frac{FL}{A Δ L}$$

where E represents the elastic modulus, σ represents the stress, ε represents the strain, F represents the compressive force, L represents the initial length of the sample, A represents the cross-sectional area of the sample, and ΔL represents the deformation amount.

As a further improvement of the present disclosure, in the step 2.3, a fixed constraint is set at a fruit stalk, and an excitation force is applied at an equatorial position. All modes within 2000 Hz are calculated, and a vibration response output is obtained through transient analysis.

Compared with the prior art, the present disclosure has the following advantages:

The bionic finite element models for the peach fruit vibration in the present disclosure fully consider a complex shape, an internal structure, and material properties of the peach fruit. The accurate 3D geometric model of the peach fruit is constructed by means of 3D scanning and reverse modeling. The elastic moduli of the peach peel, the peach flesh, the peach pit, and the peach kernel are measured through the tests and used as the material properties of the finite element models, thereby improving accuracy of finite element simulation. The present disclosure obtains the simulated dataset through the finite element simulation for transfer learning, which can realize the firmness inversion of the on-tree peach fruit based on an acoustic vibration method under a small sample size.

### BRIEF DESCRIPTION OF THE DRAWINGS

FIG. 1 is a schematic flowchart of a specific implementation according to the present disclosure;

FIG. 2 is a structural diagram of a DANN;

FIG. 3 is a structural diagram of a feature extraction network for a vibration response of a peach fruit; and

FIGS. 4A-4B show visualized features output by different networks.

### DETAILED DESCRIPTION OF THE EMBODIMENTS

The present disclosure is further described below with reference to the accompanying drawings.

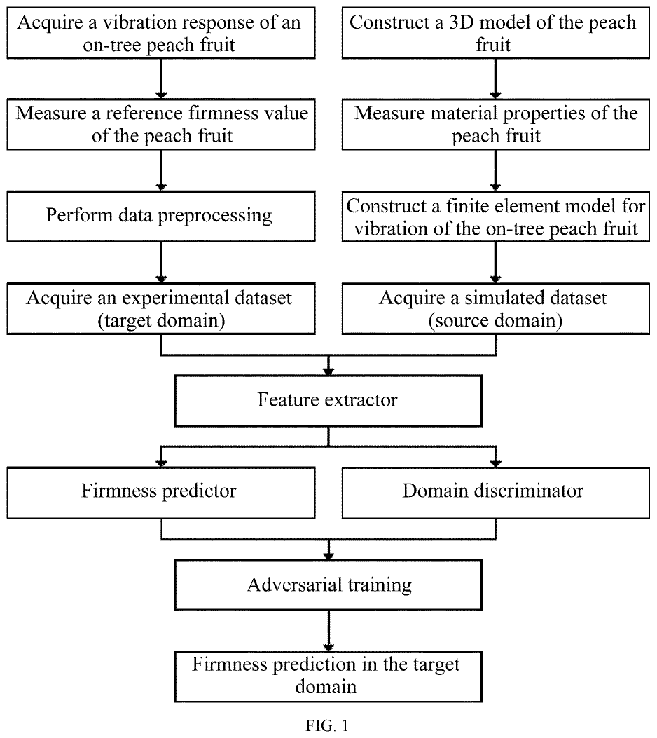

Referring to FIG. 1 to FIGS. 4A-4B, an FES and TL-based inversion method for firmness of an on-tree peach fruit includes the following steps:

Step 1: Vibration response data of on-tree peach fruits are collected, firmness values of on-tree peach fruits are measured, and an experimental dataset is constructed.

Step 1.1: Vibration signals of on-tree peach fruits at different growth stages are collected by using a laser Doppler vibrometer to obtain vibration response data of peach fruits with different firmness levels and sizes, noise reduction is performed through a band-pass filter with a cutoff frequency set to 5 Hz to 2000 Hz, and a power spectral density of a vibration response is calculated by using an autoregressive model method to serve as a model input.

Step 1.2: A puncture test is conducted on the peach fruit by using a texture analyzer with a probe diameter of 5 mm, a loading speed of 0.5 mm/s, and a loading distance of 8 mm, and an initial slope of a force-displacement curve is calculated to obtain a reference firmness value as a model output.

Step 2: Ten finite element models with high bionic fidelity for peach fruit vibration are established, and the simulated vibration responses are calculated.

Step 2.1: 3D point clouds of outer surfaces of 10 different peach fruits, peach pits, and peach kernels are separately acquired by using a handheld laser 3D scanner, and a point cloud of each part of the peach fruit is denoised and surfaced in Geomagic software, and then imported into Solidworks software to perform component combination based on a physiological structure of the peach fruit, so as to construct a complete 3D model of the peach fruit.

Step 2.2:15 peach fruits at each of the different growth stages are selected, peach flesh is prepared into a 15 mm×15 mm×15 mm cubic sample, and a compression test is conducted by using a texture analyzer with a cylindrical probe of 100 mm in diameter, a loading speed of 0.1 mm/s, and a loading distance of 8 mm. After a force-displacement curve is obtained, an elastic modulus of the peach flesh is calculated according to the following formula:

$$E = \frac{σ}{ε} = \frac{FL}{A Δ L}$$

In the above formula, E represents the elastic modulus, σ represents stress, ε represents strain, F represents a compressive force, L represents an initial length of the sample, A represents cross-sectional area of the sample, and ΔL represents a deformation amount.

A complete peach pit and peach kernel are placed on a texture analyzer with a cylindrical probe of 100 mm in diameter, a loading speed of 0.1 mm/s, and a loading distance of 3 mm for the compression test. After the force-displacement curve is obtained, elastic moduli of the peach pit and the peach kernel are calculated according to the following formula:

$$E = {\frac{0.338 {K}_{U}^{3 / 2} F ( 1 - {μ}^{2} )}{{D}^{3 / 2}} [ {{K}_{U} ( \frac{1}{{R}_{U}} + \frac{1}{{R}_{U}^{′}} )}^{1 / 3} + {{K}_{L} ( \frac{1}{{R}_{L}} + \frac{1}{{R}_{L}^{′}} )}^{1 / 3} ]}^{\frac{3}{2}}$$

In the above formula, E represents the elastic modulus, F represents a force, D represents deformation, K_{U} and K_{L} represent constants determined by curvatures of contact points on upper and lower surfaces, R_{U} and R′_{U} represent maximum and minimum curvature radii of the contact point on the upper surface, R_{L} and R′_{L} represent maximum and minimum curvature radii of the contact point on the lower surface, and curvature radii of apexes of the peach pit and the peach kernel are all provided by the 3D model.

Peach peel is prepared into a rectangular sample, and a tensile test is conducted on the peach peel by using a universal mechanical testing machine. An elastic modulus of the peach peel is calculated according to the following formula:

$$E = \frac{σ}{ε} = \frac{FL}{A Δ L}$$

In the above formula, E represents the elastic modulus, σ represents the stress, ε represents the strain, F represents the compressive force, L represents the initial length of the sample, A represents the cross-sectional area of the sample, and ΔL represents the deformation amount.

Step 2.3: The finite element models with high bionic fidelity for the peach fruit vibration are established based on a 3D geometric model of the peach fruit, material properties measured in the step 2.2, an experimental excitation, and a fruit constraint condition, a fixed constraint is set at a fruit stalk to simulate an external constraint of the peach fruit, all modes within 2000 Hz are calculated, an excitation force of 0.5 N is applied at an equatorial position to simulate a gas excitation force during the experiment, and a vibration response output is obtained through transient analysis.

Step 3: Material properties that are of the peach flesh and obtained through the finite element models in the step 2 are modified, and based on a measured elastic modulus range of the peach flesh in the test, 100 values are selected uniformly within a range of 0.5 MPa to 2.5 MPa as elastic moduli of the peach flesh to obtain simulated vibration responses of peach fruits with different firmness levels.

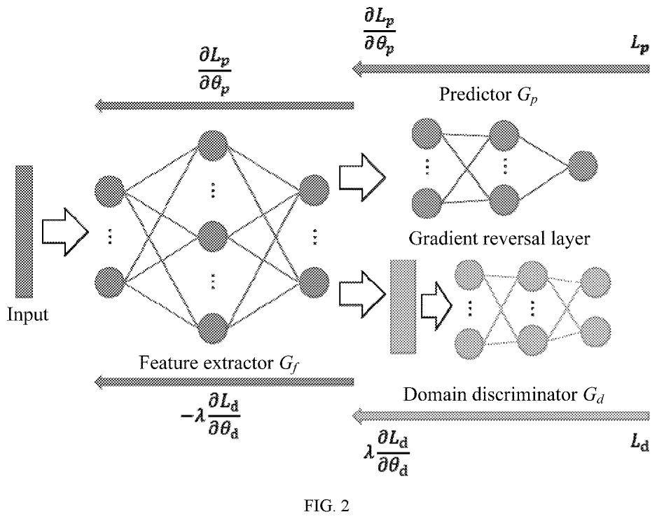

Step 4: A DANN is constructed, as shown in FIG. 2, which mainly includes a feature extraction network, a firmness predictor, a domain discriminator, and a gradient reversal layer. A gradient direction of the domain discriminator is automatically reversed through the gradient reversal layer to maximize a domain classification error and extract a domain-invariant feature. During the experiment, training is performed by using simulated data with a firmness value as a source domain and experimental data without the firmness value as a target domain, and domain transfer and firmness inversion are achieved through adversarial training.

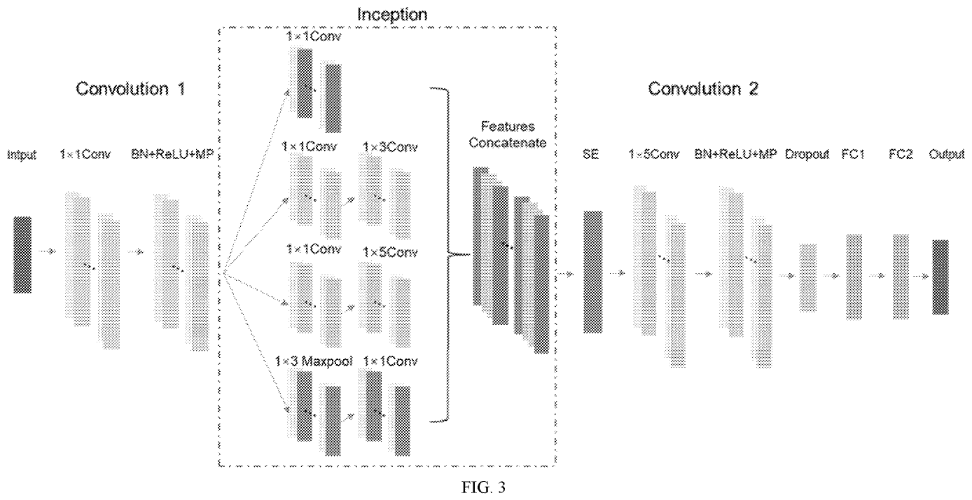

Step 4.1: The feature extraction network shown in FIG. 3 is constructed based on multi-scale convolution and a channel attention mechanism, thereby achieving multi-scale feature extraction. The feature extraction network mainly includes two convolutional layers, one Inception module, one Squeeze-and-Excitation (SE) module, and one Dropout layer. After each convolutional layer, a Batch Normalization layer, a ReLU activation function, and a maximum pooling layer are added to accelerate convergence and enhance a non-linear capability of the network.

Step 4.2: A predictor is constructed, and firmness prediction is performed on a feature through a fully connected layer. An MSE is selected as a loss function of the predictor:

$${ℒ}_{p} = \frac{{\sum }_{i = 1}^{N} {( {y}_{i} - \hat{{y}_{ι}} )}^{2}}{N}$$

In the above formula, [symbol] represents the loss function of the predictor;

N represents a quantity of samples; yᵢ represents the firmness value; and ŷ_{ι} represents a predicted firmness value.

Step 4.3: The domain discriminator is constructed. In this step, the gradient direction is automatically reversed during backpropagation through the gradient reversal layer, so as to maximize a domain classification loss and confuse target domain data and source domain data. A cross-entropy loss is used as a loss function of the domain discriminator:

$${L}_{d} = \frac{1}{N} {\sum }_{i = 1}^{N} {y}_{i} log \hat{{y}_{ι}} + ( 1 - {y}_{i} ) log ( 1 - \hat{{y}_{ι}} )$$

In the above formula, L_{d} represents the loss function of the domain discriminator; N represents the quantity of samples; yᵢ represents the firmness value; and ŷ_{ι} represents the predicted firmness value.

Step 4.4: A loss function of the network is formed jointly by the loss function of the predictor and the loss function of the domain discriminator:

L=Lₚ−λL_{d}

In the above formula, L represents a total loss function; and λ represents a parameter for adjusting an adversarial intensity.

The training is performed by using 1,000 pieces of vibration response data obtained through simulation as the source domain and using vibration response data that is of 300 peach fruits and measured experimentally as the target domain, where a learning rate is set to 0.001, penalty parameter λ is set to 1, a quantity of iterations is set to 100, and a batch size is set to 64.

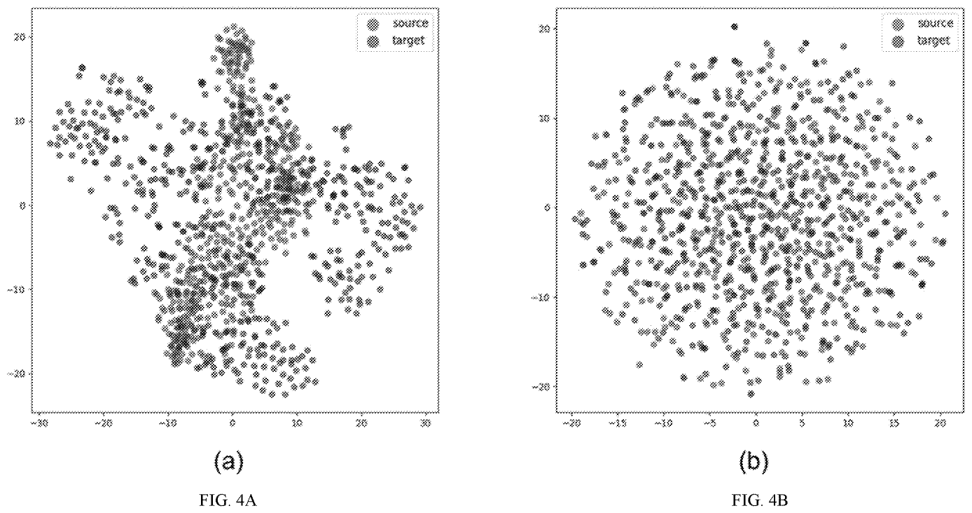

To verify effectiveness of the present disclosure, an Inception-SE network with a same network structure and an Inception-SE+DANN network are selected for comparison. Feature visualization results obtained through t-distributed stochastic neighbor embedding (t-SNE) are shown in FIG. 4A and FIG. 4B. The results indicate that the Inception-SE+DANN network in the present disclosure can effectively extract the domain-invariant feature. Effectiveness of the proposed method is evaluated using a root mean square error (RMSE) and a coefficient of determination (R²) as evaluation metrics, and results are shown in Table 1. The comparison results show that the feature extraction network proposed in the method of the present disclosure can effectively extract a feature related to firmness of the peach fruit, and the method for obtaining simulated vibration data through a finite element method and performing transfer learning can improve accuracy of firmness inversion of the on-tree peach fruit under limited samples.

TABLE 1

| Model | Input | R² | RMSE (N/mm) |
|---|---|---|---|
| CNN | Experimental data | 0.69 | 3.15 |
| Inception-SE | Experimental data | 0.79 | 2.51 |
| Inception-SE + | Experimental data and | 0.83 | 2.28 |
| DANN | simulated data | | |

What is claimed is:

1. A finite element simulation (FES) and transfer learning (TL)-based inversion method for firmness of an on-tree peach fruit, comprising following steps:
step 1, collecting vibration signals of on-tree peach fruits at different growth stages by using a laser Doppler vibrometer, preprocessing the vibration signals to serve as a model input, measuring a reference firmness value of the peach fruit by using a texture analyzer, and constructing an experimental dataset;
step 2, establishing finite element models with bionic fidelity for peach fruit vibration, and calculating a simulated vibration response;
step 3, modifying material properties of the obtained finite element models with bionic fidelity for the peach fruit vibration in the step 2 to obtain vibration responses of peach fruits with different firmness levels, and constructing a simulated dataset;
step 4, constructing a domain-adversarial neural network (DANN), performing training by using the simulated dataset obtained in the step 3 as a source domain and the experimental dataset obtained in the step 1 as a target domain, and obtaining a peach fruit firmness inversion model through adversarial training; and
step 5, inputting a vibration response of the on-tree peach fruit into the peach fruit firmness inversion model, and obtaining a firmness value of the peach fruit, so as to guide fruit picking.

2. The FES and TL-based inversion method for firmness of the on-tree peach fruit according to claim 1, wherein in the step 1, band-pass filtering and power spectral density estimation are adopted for the preprocessing.

3. The FES and TL-based inversion method for firmness of the on-tree peach fruit according to claim 1, wherein in the step 2, the establishing the finite element models with bionic fidelity for the peach fruit vibration, and calculating the simulated vibration response specifically comprises:
step 2.1: acquiring three-dimensional (3D) point clouds of outer surfaces of the peach fruit, a peach pit, and a peach kernel by using a handheld laser 3D scanner, and constructing a 3D geometric model with an accurate shape and structure of the peach fruit through reverse modeling;
step 2.2, testing compressive elastic moduli of peach flesh, the peach pit, and the peach kernel and a tensile elastic modulus of peach peel by using the texture analyzer and a universal mechanical testing machine respectively, to serve as the material properties; and
step 2.3, establishing the finite element models with bionic fidelity for the peach fruit vibration based on the 3D geometric model with the accurate shape and structure of the peach fruit and the material properties measured in the step 2.2, and simulating and calculating a simulated vibration response at a response point by using the finite element models with bionic fidelity for the peach fruit vibration under an experimental excitation and a fruit constraint condition.

4. The FES and TL-based inversion method for firmness of the on-tree peach fruit according to claim 3, wherein in the step 2.3, the experimental excitation is a gas excitation force applied to the peach fruit in an experiment.

5. The FES and TL-based inversion method for firmness of the on-tree peach fruit according to claim 3, wherein in the step 2.3, the fruit constraint condition is that the peach fruit is subjected to a constraint force at a fruit stalk.

6. The FES and TL-based inversion method for firmness of the on-tree peach fruit according to claim 1, wherein in the step 4, the constructing the DANN specifically comprises:
step 4.1, constructing a feature extraction network based on multi-scale convolution and a channel attention mechanism;
step 4.2, constructing a predictor, performing firmness prediction on a feature through a fully connected layer, and selecting a mean square error (MSE) as a loss function of the predictor:
$${ℒ}_{p} = \frac{{\sum }_{i = 1}^{{N}_{p}} {( {y}_{p} - \hat{{y}_{p}} )}^{2}}{{N}_{p}}$$
wherein [symbol] represents the loss function of the predictor; Nₚ represents a quantity of samples; yₚ represents a firmness value; and [symbol] represents a predicted firmness value;
step 4.3, constructing a domain discriminator, wherein a gradient direction is automatically reversed during backpropagation through a gradient reversal layer, and a cross-entropy loss is used as a loss function of the domain discriminator:
$${L}_{d} = \frac{1}{{N}_{d}} {\sum }_{j = 1}^{{N}_{d}} {y}_{d} log \hat{{y}_{d}} + ( 1 - {y}_{d} ) log ( 1 - \hat{{y}_{d}} )$$
wherein L_{d} represents the loss function of the discriminator; N_{d} represents the quantity of samples; y_{d} represents the firmness value; and [symbol] represents the predicted firmness value; and
step 4.4, forming a loss function of the network jointly by the loss function of the predictor and the loss function of the domain discriminator:
$$L = {L}_{p} - λ {L}_{d}$$
wherein L represents a total loss function; and λ represents a parameter for adjusting an adversarial intensity.
